SEQUENCE LISTING (1 sequence)

SEQ ID NO: 1 - protein, 5 aa, Artificial Sequence
  MOD_RES (5): AMIDATION
YIGSR

(19) United States
(12) Patent Application Publication
(10) Pub. No.: US 2004/0180381 A1
(43) Pub. Date: Sep. 16, 2004

(54) Method for determining amino acid sequence of a peptide

(76) Inventors: Minoru Yamaguchi, Kyoto-shi (JP); Eiji Ando, Kyoto-shi (JP); Shigemi Norioka, Osaka (JP); Norikazu Ueyama, Osaka (JP); Taka-aki Okamura, Osaka (JP); Takashi Nakazawa, Nara-shi (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 10/796,062

(22) Filed: Mar. 10, 2004

(30) Foreign Application Priority Data
Mar. 11, 2003 (JP) PAT.2003-64735

(51) Int. Cl. G01N 33/53; C07K 14/00

(52) U.S. Cl. 435/7.1; 530/409

(57) ABSTRACT

The present invention provides a method for determining amino acid sequences of peptides involving the use of a mass spectrometer that not only offers a high decomposition efficiency into fragment ions and a high selectivity of cleavage sites for a wide variety of peptide including peptides of unknown identity, but also achieves highly sensitive, high-throughput detection of the resulting fragment ions. A method for determining amino acid sequence of a peptide, comprising the steps of: preparing a peptide of interest or fragments thereof obtained by optionally cleaving the peptide of interest; coupling an amino acid derivative to the N-terminus of the peptide of interest or the fragments thereof, the amino acid derivative having protected an amino group with a protective group and derived from an amino acid with a side chain containing an acidic group; and subjecting the coupled product to mass spectrometry analysis.

### BACKGROUND OF THE INVENITION

1. Field of the Invention

The present invention relates to a method for determining amino acid sequences of peptides.

2. Disclosure of the Related Art

Among conventional mass-spectroscopic techniques for determining amino acid sequences of peptides are MS/MS analysis using Post-Source Decay technique (PSD) and MS/MS analysis using ESI-Q-TOF mass spectrometry. In PSD analysis, on a MALDI-TOF mass spectrometer, a peptide of interest is ionized and the generated ions (i.e., precursor ions) undergo spontaneous decomposition during flight into different PSD ions, which in turn are separated and detected. In each of these techniques, precursor ions resulting from a peptide are selected and are decomposed on a mass spectrometer into product ions. The resulting peptide fragments are analyzed to provide information on the amino acid sequence of the peptide. In these techniques, however, peptide fragmentation takes place not only in peptide bonds but also in sites other than peptide bonds, resulting in a complex mixture of peptide fragments. This results in mixed spectra of those different fragments, as well as spectra of fragment containing the N-terminus of the peptide of interest and fragment containing the C-terminus of the peptide of interest. Such spectra are generally complicated and difficult to analyze. Though the recent development of search engines such as “Mascot” (http://www.matrixscience.com/) has enabled database searches to determine the peptide sequence from such complicated spectrum patterns, the number of identifiable peptides has been limited since reference can be made only to the peptides available on these databases.

In one approach described in Japanese Patent Laid-Open Publication No. Hei 10-90226, the N-terminus and the C-terminus of a peptide are chemically modified in order to facilitate the generation of product ions and thereby simplify the resulting peptide fragments. In this manner, the peptide fragments containing modified termini can be detected at high sensitivity in MS/MS analysis and the amino acid sequence can be directly determined from the difference in the molecular weights of the peptide fragments.

### SUMMARY OF THE INVENTION

Relying on chemical modification, however, this approach has disadvantages that the efficiency of the decomposition into product ions is limited, that the selectivity of the cleavage site is insufficient, and that the efficiency of the decomposition into prooduct ions, as well as the selectivity of the cleavage sites, can vary significantly depending on the internal sequences of the peptide, limiting the number of identifiable peptides.

Accordingly, it is an objective of the present invention to provide a method for determining amino acid sequences of peptides involving the use of a mass spectrometer that not only offers a high decomposition efficiency into product ions and a high selectivity of cleavage sites for a wide variety of peptide including peptides of unknown identity, but also achieves highly sensitive, high-throughput detection of the resulting product ions.

In the course of our studies, the present inventors have discovered that the above-described objective of the present invention can be attained by coupling an amino acid derivative to the N-terminus of a peptide of interest or fragments thereof obtained by optionally cleaving the peptide of interest, the amino acid derivative having an amino group protected with a protective group and a side-chain organic group with an acidic group, and subjecting the resulting product to PSD method or MS/MS analysis. The discovery inspired the present inventors to ultimately devise the present invention.

The present invention comprises the following inventions:

(1) A method for determining amino acid sequence of a peptide, comprising the steps of:

preparing a peptide of interest or fragments thereof obtained by optionally cleaving the peptide of interest;

coupling an amino acid derivative to the N-terminus of the peptide of interest or the fragments thereof, the amino acid derivative having protected an amino group with a protective group and derived from an amino acid with a side chain containing an acidic group; and

subjecting the coupled product to mass spectrometry analysis.

As used herein, the term “peptide” is meant to encompass proteins.

(2) The method according to the above (1), wherein the acidic group is selected from the group consisting of carboxyl, sulfo, phosphono, sulfate, and phosphate group.

(3) The method according to the above (1) or (2), wherein the amino acid is selected from the group consisting of cysteic acid, aspartic acid, glutamic acid, threonine phosphate, serine phosphate, tyrosine sulfate, and tyrosine phosphate.

(4) The method according to any one of the above (1) to (3), wherein the protective group is a functional group other than a basic group.

(5) The method according to any one of the above (1) to (3), wherein the protective group is selected from the group consisting of biotinyl, acetyl, formyl, and phenylisothiocarbamyl.

(6) The method according to any one of the above (1) to (3), wherein the protective group is biotinyl.

(7) The method according to the above (1), wherein the amino acid derivative is N-biotinylcysteic acid.

(8) The method according to any one of the above (1) to (7), wherein the cleaving is performed by using an enzyme that can specifically hydrolyze a peptide bond on the C-terminal side of a basic amino acid residue.

(9) The method according to any one of the above (1) to (8), wherein the peptide or the peptide fragment coupled to the amino acid derivative is ionized and is decayed into decay ions, which are then subjected to mass spectrometry for separation and detection.

(10) The method according to the above (9), wherein the peptide or the peptide fragment coupled to the amino acid derivative is ionized by matrix-assisted laser desorption inonization (MALDI).

(11) The method according to the above (9) or (10), wherein the ions are separated and detected by time-of-flight mass spectrometry (TOFMS).

According to the present invention, there is provided a method for determining amino acid sequences of peptides involving the use of a mass spectrometer that not only offers a high decomposition efficiency into fragment ions and a high selectivity of cleavage sites for a wide variety of peptide including peptides of unknown identity, but also achieves highly sensitive, high-throughput detection of the resulting fragment ions.

### BRIEF DESCRIPTION OF THE DRAWINGS

FIG. 1 shows PSD spectrum of laminin pentapeptide having the N-terminus bound to N-biotinylcysteic acid.

FIG. 2 shows PSD spectrum of laminin pentapeptide in which the N-terminus is not bound to N-biotinylcysteic acid.

### DETAILED DESCRIPTION OF THE INVENTION

The method for determining amino acid sequences of peptides in accordance with the present invention involves optionally cleaving a peptide of interest into fragments thereof and preparing a peptide of interest or the fragments thereof; coupling an amino acid derivative to the N-terminus of the peptide of interest or the fragment thereof; and subjecting the resulting coupled product to mass spectrometry analysis.

The amino acid derivative for use in the present invention may be derived from any of an α-amino acid, β-amino acid, γ-amino acid, and δ-amino acid.

According to the present invention, when the peptide of interest is a large molecule such as a protein, it is preferably to cleave into smaller fragments. The fragmentation is preferably done by enzymatic digestion. When used, the enzyme is preferably one that can specifically hydrolyze apeptide bond on the C-terminal side of a basic amino acid residue. Examples of such enzymes include trypsin, plasmin, thrombin, lysyl endopeptidase, and arginine endopeptidase. Thus, the fragmentation results in peptide fragments having a basic amino acid residue at the C-terminal. When the resulting peptide fragments are placed in water, the basic C-terminal residue of the peptide fragment is protonated so that the peptide fragment bears a positive charge. This property of the peptide fragments is favorable in that, during the ionization of the peptide fragments and the generation of fragment ions on a mass spectrometer, C-terminal residue is positively charged by protonation, resulting in facilitating generation of the product ions having such a C-terminal residue.

According to the present invention, an amino acid derivative is coupled to the N-terminus of a peptide of interest or, when the peptide is cleaved into fragments, a fragment thereof. The amino acid derivative is one having protected an amino group with a protective group and derived from an amino acid with a side chain containing an acidic group. When such an amino acid derivative is placed in water, a proton dissociates from the acidic group and, as a result, the amino acid derivative becomes negatively charged. The coupling of the negatively charged amino acid derivative to the N-terminus of the peptides or the peptide fragments facilitates the generation of the positively charged product ions.

Examples of the acidic group include carboxyl (CO₂H), sulfo (SO₃H), phosphono (PO₃H₂), sulfate (OSO₃H), and phosphate (OPO₃H₂).

Thus, the amino acid derivative may derived from, for example, acidic amino acids, such as cysteic acid, aspartic acid, and glutamic acid; and hydroxyl-containing amino acids with their hydroxyl group sulfated or phosphorylated, such as threonine phosphate, serine phosphate, tyrosine sulfate, and tyrosine phosphate; and the like.

While, in theory, the protective group may be any protective group that does not allow the molecule of the amino acid derivative to lose, as a whole molecule, its negative charge in water, the protective group is preferably a functional group other than a basic group, namely a functional group that does not include a basic group since a basic group may neutralize the charge of the acidic group, thus canceling out the charge of the amino acid derivative as a whole molecule. Specific examples of the functional groups that do not include a basic group include biotinyl, acetyl, formyl, and phenylisothiocarbamyl. Of these, biotinyl group is particularly preferred in the present invention.

According to the present invention, the above-described protective group serves to prevent the amino group of the amino acid derivative from becoming positively charged and thereby, when the peptide of interest coupled with the amino acid derivative is subjected to mass spectrometry, facilitate the generation of positively charged product ions that are not bound to the amino acid derivative. If the amino acid derivative is not protected with the protective group, the positive charge on the amino group of the amino acid derivative may cancel out with the negative charge of the side chain, thus interfering with the generation of such positively charged product ions as described above.

A particularly preferred amino acid derivative, that has its amino group protected by the above-described protective group and has a side-chain organic group having an acidic group, is N-biotinylcysteic acid of the following chemical formula (I):

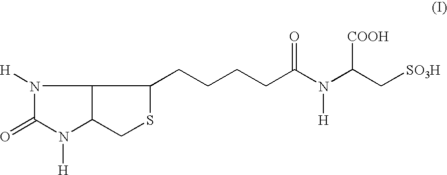

According to the present invention, known conventional peptide synthesis techniques may be used to couple the above-described amino acid derivative to the N-terminus of the peptide fragments: either of the liquid phase synthesis or the solid phase synthesis may be used to bind the amino acid derivative. Using these techniques, a peptide or a peptide fragment coupled to the amino acid derivative may be obtained (referred to simply as ‘peptide molecule,’ hereinafter). The peptide molecules so obtained are then subjected to mass spectrometry for analysis.

While the peptide molecules may be ionized using any proper technique, the ionization may be carried out by using such techniques as matrix-assisted laser desorption inonization (MALDI), electrospray ionization (ESI), fast atom bombardment (FAB), liquid secondary ion mass spectrometry (LSIMS), and liquid ionization (LI) and the like.

Each of MALDI, FAB, LSIMS, and LI involves the use of matrix to absorb laser light, so that the laser light does not need to be directly absorbed by the peptide molecules. For this reason, each of these techniques may be suitably used to ionize a wide range of compounds. MALDI is a particularly preferred technique for use in the present invention because the technique offers the following advantageous features: (i) it allows instant ionization (pulsed ionization); (ii) it can achieve highly efficient ionization; (iii) it allows ionization of a wide range of compounds; and (iv) it allows ionization of an unpurified compound or a mixture of compounds.

ESI is also preferred since peptide molecules can be ionized without being destroyed and are formed into product ions.

In the present invention, it is preferred to ionize peptide molecules, allow the resulting ions to decay into product ions, and then subject these ions to mass spectrometry for separation and detection. Specifically, peptide molecules are preferably ionized using MALDI to form precursor ions, which subsequently undergo decomposition into product ions. The product ions are then subjected to time-of-flight mass spectrometry (TOFMS) for separation and detection. In TOFMS, ions of different masses were separated based on the time of flight, and high-sensitivity high-resolution measurement can be carried out. Therefore, as TOFMS is compatible with MALDI in which ions are generated in pulsed form, combination thereof (i.e. MALDI-TOFMS) is one of preferable technique.

Accordingly, it is particularly preferred in the present invention that the ionization of the peptide molecules be carried out using MALDI and the separation/detection of the product ions generated by PSD using TOFMS.

According to the present invention, the MS/MS analysis using the above-described amino acid derivative and conducted based on preferable MALDI TOF MS allows selective and highly sensitive detection of peptide fragments containing the C-terminus of a peptide of interest (y series) and also allows easy determination of the amino acid sequence of the peptide from the difference in the molecular weights of the peptide fragments.

### EXAMPLES

The present invention will now be described in further detail with reference to Examples, which are provided by way of example only and are not intended to limit the scope of the invention in any way.

### Example 1

(1) Synthesis of N-biotinylcysteic Acid

3.4 mg sulfosuccinimide biotin dissolved in 20 μl distilled water, 1.1 mg cysteic acid dissolved in 15 μl distilled water, and 1.65 μl triethylamine were mixed with one another and the reaction was allowed to proceed at 60° C. for 30 min. The reaction product was purified by reverse phase HPLC and was identified as the aimed N-biotinylcysteic acid using MALDI-TOF MS.

(2) Coupling of N-Biotinylcysteic Acid to Peptide

As a model peptide, laminin pentapeptide (PEPTIDE INSTITUTE, Inc.) was used. The amino acid sequence of the laminin pentapeptide is as follows: Tyr-Ile-Gly-Ser-Arg-NH₂. In this sequence, the arginine residue is amidated and is represented as Arg-NH₂. 2 μl of 1 mM dimethylformamide solution of N-biotinylcysteic acid, 0.6 μl of dimethylformamide solution containing 0.5M HBTU (2-[1H-benzotriazole-1-yl]-1,1,3,3-tetramethyluronium hexafluorophosphate) and 0.5M HOBt (N-hydroxybenzotriazole), and 0.6 μl of 1M dimethylformamide solution of diisopropylethylamine were mixed with one another. The mixture was then added to 2 μl of 2 mM dimethylformamide solution of laminin pentapeptide, and the reaction was allowed to proceed at room temperature for 30 min. After the reaction was completed, the reaction mixture was diluted with 0.1 w % aqueous solution of trifluoroacetic acid and was subjected to PSD analysis by MALDI-TOF MS.

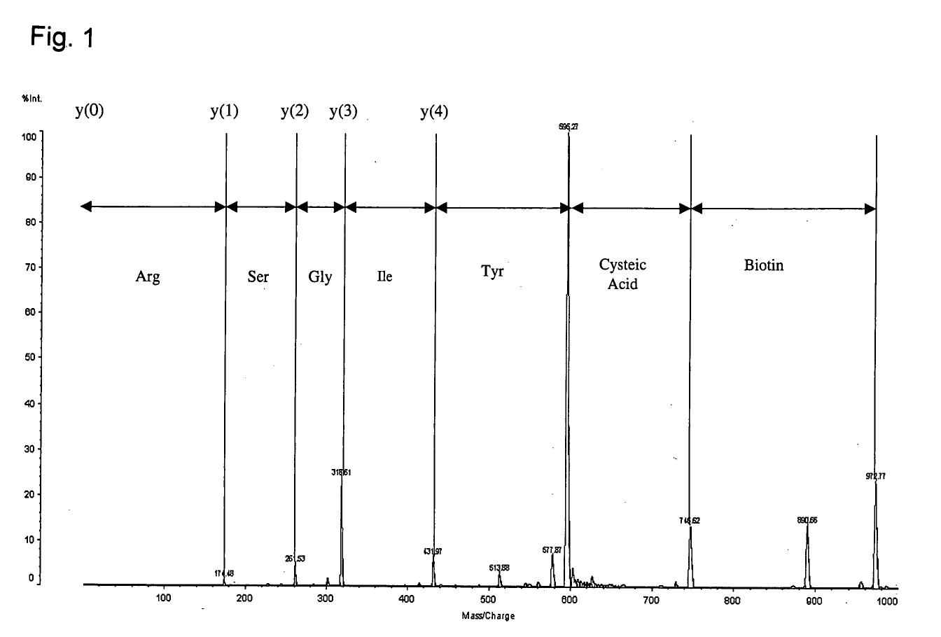

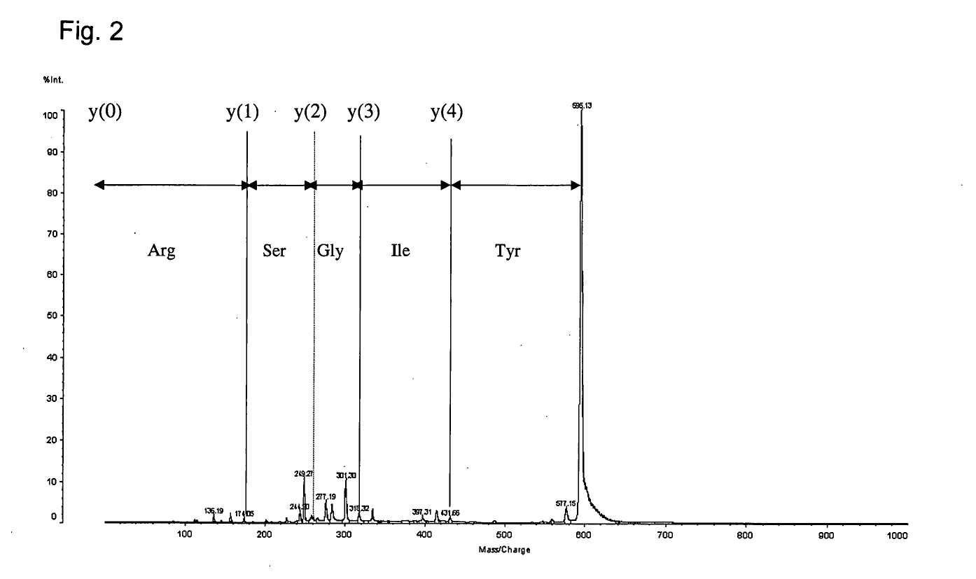

FIG. 1 shows the PSD spectra of laminin pentapeptide that is coupled to N-ciotinylcysteic acid. FIG. 2 shows the PSD spectra of laminin pentapeptide that is not coupled to N-biotinylcysteic acid. In each figure, horizontal axis indicates the mass-to-charge ratio of the ions (m/z), whereas vertical axis indicates the relative intensity of the ions (Int.). A single alphabet letter followed by a parenthesized number shown above each peak of the decay products indicates the position of cleaved peptide bonds. The letter y signifies that the peptide is derived from the. C-terminus and each number indicates the number of remaining amino acid residues.

As shown in FIG. 1, biotin and cysteic acid derived from N-biotinylcysteic acid bound to laminin pentapeptide are shown dissociated. In FIG. 1, y-series fragments of the peptide molecules are detected at higher selectivity and higher sensitivity than in the results of FIG. 2 in which laminin pentapeptide was not modified with biotinylcysteic acid.

According to the present invention, there has been provided a novel method for determining amino acid sequences of peptides through the use of a mass spectrometer. Not only does this method offer a high decomposition efficiency into fragment ions and a high selectivity of cleavage sites for a wide spectrum of peptides including peptides of unknown identity, but it also achieves highly sensitive, high-throughput detection of the resulting fragment ions.

The above-described Example shows a concrete mode within the scope of the present invention, however, the present invention can be carried out in various other modes. Therefore, the above-described Example is merely illustrative in all respects, and must not be construed. Further, the changes that fall within the equivalents of the claims are all within the scope of the present invention.

What is claimed is:

1. A method for determining amino acid sequence of a peptide, comprising the steps of:
preparing a peptide of interest or fragments thereof obtained by optionally cleaving the peptide of interest;
coupling an amino acid derivative to the N-terminus of the peptide of interest or the fragments thereof, the amino acid derivative having protected an amino group with a protective group and derived from an amino acid with a side chain containing an acidic group; and
subjecting the coupled product to mass spectrometry analysis.

2. The method according to claim 1, wherein the acidic group is selected from the group consisting of carboxyl, sulfo, phosphono, sulfate, and phosphate group.

3. The method according to claim 1, wherein the amino acid is selected from the group consisting of cysteic acid, aspartic acid, glutamic acid, threonine phosphate, serine phosphate, tyrosine sulfate, and tyrosine phosphate.

4. The method according to claim 1, wherein the protective group is a functional group other than a basic group.

5. The method according to claim 1, wherein the protective group is selected from the group consisting of biotinyl, acetyl, formyl, and phenylisothiocarbamyl.

6. The method according to claim 1, wherein the protective group is biotinyl.

7. The method according to claim 1, wherein the amino acid derivative is N-biotinylcysteic acid.

8. The method according to claim 1, wherein the cleaving is performed by using an enzyme that can specifically hydrolyze a peptide bond on the C-terminal side of a basic amino acid residue.

9. The method according to claim 1, wherein the peptide or the peptide fragment coupled to the amino acid derivative is ionized and is decayed into decay ions, which are then subjected to mass spectrometry for separation and detection.

10. The method according to claim 9, wherein the peptide or the peptide fragment coupled to the amino acid derivative is ionized by matrix-assisted laser desorption inonization (MALDI).

11. The method according to claim 9, wherein the ions are separated and detected by time-of-flight mass spectrometry (TOFMS).
